SEQUENCE LISTING (39 sequences)

SEQ ID NO: 1 - DNA, 1362 bp, Hepatitis X Related Virus
actaccaacagatcctcgacgaactgcgccaggaactggccgagcactacctgctgcgcagcgacctggcgatccaggatatcgcctgctacctcggtttcaccgagtcacgctcgttccaccgcagtttcaagagctggaccgggcagacgccgggcgagtttcgcgagagccggcgccgggataatccgctgggctagcgcgatatggccggaaacgccgtgccagccagtagtcgagactcaaccatcgccccgccccgctgatgaaaagcgccacgagcgcagccacggccggcaccggtgaggtttgccaatggcatatcagtcctcccggcgcccttactcgttcttatcgccactgcacgtgccttcaatacgggagccttcctgcgccttctcggcagcggtcaggctgtagccgccggccagttcctgctcagcgaaggggatgctagtggcgtgggcagtgaacgccatgtaactcggcagtgcagcgccctagggtctgttgccgtttcgcgcacggccgcgtcgaaacggcaacagaccctaggtggcagtcagggtattggcatctctccatcggtttcgaatacggcgccaggttggcgccctcgcagcaatggacgaggcagggatgcgggcgttacagcgggcgaaaaagatttctcgtagcccgatgaaatacgggggcgctttgctcgccagcaatcgcggctacgactgcatggacgcaggaggtagagcgaagcaggatgvvagagcagaaagctctctcccacagacacagaaacatccaccgcacggtaggaggtgattcaaatgatcaggcatctcctctggttggactgcatggccgctgcgagcacgggcgttgtggttctgttgctggcccccctggttgagcggctggtatgccctgcccggcgagctgctgagcttcatcggcgcgatcaatatcgcctacgcctgcttttccatttcgctggcgattcgcctgcgacgcgccgaagcgctaatcaagctgctggcagtggccaacggactctgggcgttggcatgccttggcatcgctacgatctttgccccgctcatgacgctaccggggctttgtcatgtgctcggcgaggctgcatccgtcgcaggcctgggcatgctggagtggaaatggcgcaggcagctgctggtggctggcgaacagggcgtttcgactcagcttgtcgcggtccagtaaccgtcacaggtattcaggcgaagatccggcgatggctgttcaggccatcatcagccctatccttcagccctgtgaaagcggttcttgcccgcgtgcttggccgcgtacctcggccccgaccacgct

SEQ ID NO: 2 - DNA, 562 bp, Hepatitis X Related Virus
tagtcgagactcaaccatcgctcccgccccgctgatgaaaaggtcgctcggctcaagcgcgaactgtcgcgtgttaccaaggaacgagattttttacgagacgcggcagcgtacttcgcgaagcaatcgccgaacggtacgcggtgatcgagcgctgccgcagcgactaccccattgggatgatgtgccgctgccttcaagtgtcgaccagtgggttctacgcctgggccaggcgaaagccggggccgcgtgcccaggcgaattcgcgtctcttggagcgcatgcgtgaaatccacgaggacagccgaggcatcatcggcgcgcgtcggatgcaggaagacctcgccgacgaaggcatgcccgccagcttgaatcgggtggcccgcgtcatggccaaggccgggcttcagggctggccgcggcgaaagaagcgtggctttccgcgcaagccgccgacgcgtcgtcccgagggcgtcaggaaccttctggagagggatttctcggcgctcgaaccggagacgaagtgggtaaccgacatcaccgagatcgtcaccgacgaggg

SEQ ID NO: 3 - DNA, 571 bp, Hepatitis X Related Virus
taccagaagttcctcggcagccatgccttggtctgcagcatgagcgaggtcggccattgcggcgacaacgcagcatgtgagggattcttcgggctactgaagcgagagtggatctaccaaacccgctacagcacaagaagggaagctcgggccgacgtctttgcctacctggagcggttccacgacccgcggatgcgccgtagagttgcgaggcgtgaccgggagtttcaagccttaatcaaaccgtccgcggaaacggggtagaacccgagtccacttaccgccggtgcggcgcaggtcgccgccccacaccacgcaggttaagtcgagttccgaaccctgcacctgaaactcagtggcgacgtcttcgaggtagtacgacgtcttcgcggtgatacaagaaacgtgtgtcctccttgccggccaaaagccactggataaggtcgacctttaagcgcattcccatagcgtactgggacccctgatgccgaggcacgaagtagcaggtaacatcgtgtcatgcacaagcaatcggatcatgtcgtctcgctcttttcatgagcggggcggg

SEQ ID NO: 4 - DNA, 1314 bp, Hepatitis X Related Virus
tagtcgagactcaaccatcgctcccgccccgctgatgaaaaggtcgctcggctcaagcgcgaactgtcgcgtgttaccaaggaacgagattttttacgagacgcggcagcgtacttcgcgaagcaatcgccgaacggtacgcggtgatcgagcgctgccgcagcgactaccccattgggatgatgtgccgctgccttcaagtgtcgaccagtgggttctacgcctgggccaggcgaaagccggggccgcgtgcccaggcgaattcgcgtctcttggagcgcatgcgtgaaatccacgaggacagccgaggcatcatcggcgcgcgtcggatgcaggaagacctcgccgacgaaggcatgcccgccagcttgaatcgggtggcccgcgtcatggccaaggccgggcttcagggctggccgcggcgaaagaagcgtggctttccgcgcaagccgccgacgcgtcgtcccgagggcgtcaggaaccttctggagagggatttctcggcgctcgaaccggagacgaagtgggtaaccgacatcaccgagatcgtcaccgacgagggaaaactccatctctgcgtcgtcctcgacctgtacagcaaactcatcatgggatggtcgatgcatcaccggcaggatcgccacatggtggttcgcgcggtacagatggcggtttggcagcgcgagggcggcgacgaggtgatcctgcattccgatcgcggcgggcagttcatcagcgatacgtaccagaagttcctcggcagccatgccttggtctgcagcatgagcgaggtcggccattgcggcgacaacgcagcatgtgagggattcttcgggctactgaagcgagagtggatctaccaaacccgctacagcacaagaagggaagctcgggccgacgtctttgcctacctggagcggttccacgacccgcggatgcgccgtagagttgcgaggcgtgaccgggagtttcaagccttaatcaaaccgtccgcggaaacggggtagaacccgagtccacttaccgccggtgcggcgcaggtcgccgccccacaccacgcaggttaagtcgagttccgaaccctgcacctgaaactcagtggcgacgtcttcgaggtagtacgacgtcttcgcggtgatacaagaaacgtgtgtcctccttgccggccaaaagccactggataaggtcgacctttaagcgcattcccatagcgtactgggacccctgatgccgaggcacgaagtagcaggtaacatcgtgtcatgcacaagcaatcggatcatgtcgtctcgctcttttcatgagcggggcggg

SEQ ID NO: 5 - DNA, 285 bp, Hepatitis X Related Virus
agtcgagactcaaccatcgctcccgccccgctgatgaaaaggtcgctcggctcaagcgcgaactgtcgcgtgttaccaaggaacgagattttttacgagacgcggcagcgtacttcgcgaagcaatcgccgaacggtacgcggtgatcgagcgctgccgcagcgactaccccattgggatgatgtgccgctgccttcaagtgtcgaccagtgggttctacgcctgggccaggcgaaagccggggccgcgtgcccaggcgaattcgcgtctcttggagcgcatgcg

SEQ ID NO: 6 - DNA, 141 bp, Hepatitis X Related Virus
tcgagactcaaccatcgctcccgccccgctgatgaaaaggtcgctcggctcaagcgcgaactgtcgcgtgttaccaaggaacgagattttttacgagacgcggcagcgtacttcgcgaagcaatcgccgaacggtacgcgg

SEQ ID NO: 7 - DNA, 825 bp, Hepatitis X Related Virus
atgatgtgccgctgccttcaagtgtcgaccagtgggttctacgcctgggccaggcgaaagccggggccgcgtgcccaggcgaattcgcgtctcttggagcgcatgcgtgaaatccacgaggacagccgaggcatcatcggcgcgcgtcggatgcaggaagacctcgccgacgaaggcatgcccgccagcttgaatcgggtggcccgcgtcatggccaaggccgggcttcagggctggccgcggcgaaagaagcgtggctttccgcgcaagccgccgacgcgtcgtcccgagggcgtcaggaaccttctggagagggatttctcggcgctcgaaccggagacgaagtgggtaaccgacatcaccgagatcgtcaccgacgagggaaaactccatctctgcgtcgtcctcgacctgtacagcaaactcatcatgggatggtcgatgcatcaccggcaggatcgccacatggtggttcgcgcggtacagatggcggtttggcagcgcgagggcggcgacgaggtgatcctgcattccgatcgcggcgggcagttcatcagcgatacgtaccagaagttcctcggcagccatgccttggtctgcagcatgagcgaggtcggccattgcggcgacaacgcagcatgtgagggattcttcgggctactgaagcgagagtggatctaccaaacccgctacagcacaagaagggaagctcgggccgacgtctttgcctacctggagcggttccacgacccgcggatgcgccgtagagttgcgaggcgtgaccgggagtttcaagccttaatcaaaccgtccgcggaaacgggg

SEQ ID NO: 8 - DNA, 207 bp, Hepatitis X Related Virus
atggtcgatgcatcaccggcaggatcgccacatggtggttcgcgcggtacagatggcggtttggcagcgcgagggcggcgacgaggtgatcctgcattccgatcgcggcgggcagttcatcagcgatacgtaccagaagttcctcggcagccatgccttggtctgcagcatgagcgaggtcggccattgcggcgacaacgcagcatg

SEQ ID NO: 9 - DNA, 87 bp, Hepatitis X Related Virus
atgccgaggcacgaagtagcaggtaacatcgtgtcatgcacaagcaatcggatcatgtcgtctcgctcttttcatgagcggggcggg

SEQ ID NO: 10 - DNA, 87 bp, Hepatitis X Related Virus
agcgcattcccatagcgtactgggacccctgatgccgaggcacgaagtagcaggtaacatcgtgtcatgcacaagcaatcggatcat

SEQ ID NO: 11 - DNA, 198 bp, Hepatitis X Related Virus
agtcgagttccgaaccctgcacctgaaactcagtggcgacgtcttcgaggtagtacgacgtcttcgcggtgatacaagaaacgtgtgtcctccttgccggccaaaagccactggataaggtcgacctttaagcgcattcccatagcgtactgggacccctgatgccgaggcacgaagtagcaggtaacatcgtgtcat

SEQ ID NO: 12 - DNA, 111 bp, Hepatitis X Related Virus
gtggcgacgtcttcgaggtagtacgacgtcttcgcggtgatacaagaaacgtgtgtcctccttgccggccaaaagccactggataaggtcgacctttaagcgcattcccat

SEQ ID NO: 13 - DNA, 84 bp, Hepatitis X Related Virus
gcgatacgtaccagaagttcctcggcagccatgccttggtctgcagcatgagcgaggtcggccattgcggcgacaacgcagcat

SEQ ID NO: 14 - DNA, 795 bp, Hepatitis X Related Virus
gagactcaaccatcgctcccgccccgctgatgaaaaggtcgctcggctcaagcgcgaactgtcgcgtgttaccaaggaacgagattttttacgagacgcggcagcgtacttcgcgaagcaatcgccgaacggtacgcggtgatcgagcgctgccgcagcgactaccccattgggatgatgtgccgctgccttcaagtgtcgaccagtgggttctacgcctgggccaggcgaaagccggggccgcgtgcccaggcgaattcgcgtctcttggagcgcatgcgtgaaatccacgaggacagccgaggcatcatcggcgcgcgtcggatgcaggaagacctcgccgacgaaggcatgcccgccagcttgaatcgggtggcccgcgtcatggccaaggccgggcttcagggctggccgcggcgaaagaagcgtggctttccgcgcaagccgccgacgcgtcgtcccgagggcgtcaggaaccttctggagagggatttctcggcgctcgaaccggagacgaagtgggtaaccgacatcaccgagatcgtcaccgacgagggaaaactccatctctgcgtcgtcctcgacctgtacagcaaactcatcatgggatggtcgatgcatcaccggcaggatcgccacatggtggttcgcgcggtacagatggcggtttggcagcgcgagggcggcgacgaggtgatcctgcattccgatcgcggcgggcagttcatcagcgatacgtaccagaagttcctcggcagccatgccttggtctgcagcatgagcgaggtcggccat

SEQ ID NO: 15 - DNA, 156 bp, Hepatitis X Related Virus
ccggcaggatcgccacatggtggttcgcgcggtacagatggcggtttggcagcgcgagggcggcgacgaggtgatcctgcattccgatcgcggcgggcagttcatcagcgatacgtaccagaagttcctcggcagccatgccttggtctgcagcat

SEQ ID NO: 16 - DNA, 201 bp, Hepatitis X Related Virus
gggctggccgcggcgaaagaagcgtggctttccgcgcaagccgccgacgcgtcgtcccgagggcgtcaggaaccttctggagagggatttctcggcgctcgaaccggagacgaagtgggtaaccgacatcaccgagatcgtcaccgacgagggaaaactccatctctgcgtcgtcctcgacctgtacagcaaactcatcat

SEQ ID NO: 17 - DNA, 171 bp, Hepatitis X Related Virus
cgcctgggccaggcgaaagccggggccgcgtgcccaggcgaattcgcgtctcttggagcgcatgcgtgaaatccacgaggacagccgaggcatcatcggcgcgcgtcggatgcaggaagacctcgccgacgaaggcatgcccgccagcttgaatcgggtggcccgcgtcat

SEQ ID NO: 18 - protein, 95 aa, Hepatitis X Related Virus
SRDSTIAPAPLMKRSLGSSANCRVLPRNEIFYETRQRTSRSNRRTVRGDRALPQRLPHWDDVPLPSSVDQWVLRLGQAKAGAACPGEFASLGAHA

SEQ ID NO: 19 - protein, 47 aa, Hepatitis X Related Virus
SRLNHRSRPADEKVARLKRELSRVTKERDFLRDAAAYFAKQSPNGTR

SEQ ID NO: 20 - protein, 275 aa, Hepatitis X Related Virus
MMCRCLQVSTSGFYAWARRKPGPRAQANSRLLERMREIHEDSRGIIGARRMQEDLADEGMPASLNRVARVMAKAGLQGWPRRKKRGFPRKPPTRRPEGVRNLLERDFSALEPETKWVTDITEIVTDEGKLHLCVVLDLYSKLIMGWSMHHRQDRHMVVRAVQMAVWQREGGDEVILHSDRGGQFISDTYQKFLGSHALVCSMSEVGHCGDNAACEGFFGLLKREWIYQTRYSTRREARADVFAYLERFHDPRMRRRVARRDREFQALIKPSAETG

SEQ ID NO: 21 - protein, 69 aa, Hepatitis X Related Virus
MVDASPAGSPHGGSRGTDGGLAARGRRRGDPAFRSRRAVHQRYVPEVPRQPCLGLQHERGRPLRRQRSM

SEQ ID NO: 22 - protein, 29 aa, Hepatitis X Related Virus
MPRHEVAGNIVSCTSNRIMSSRSFHERGG

SEQ ID NO: 23 - protein, 29 aa, Hepatitis X Related Virus
RCEWLTSPGRIGLCSTAPLMTDHVLLRIM

SEQ ID NO: 24 - protein, 66 aa, Hepatitis X Related Virus
TSNRVRCRFSLPSTKSTTRRRRPSVLFTHGGQRGFAVPYPRGKLANGYRVPVGSASARLLLYCRTM

SEQ ID NO: 25 - protein, 37 aa, Hepatitis X Related Virus
HRRRRPLVVDERHYLFRTDEKGALLWQILDVKLRMGM

SEQ ID NO: 26 - protein, 28 aa, Hepatitis X Related Virus
RYTGSTGRCGHRPRCCSRPRGNRRCRLM

SEQ ID NO: 27 - protein, 265 aa, Hepatitis X Related Virus
LSLWRERGASSFTARSLRSSDRTVLSRSKKRSAAAYKAFCDGFPVRHDLAAAQRLPHWDDVPLPSSVDQWVLRLGQAKAPGRAWAFERRKSRMRSIWSSLRPMMPARRMQEDLADEGMPASLNRVARVMAKAGLQGWPRRKKRGFPRKPPTRRPEGVRNLLERDFSALEPETKWVTDITEIVTDEGKLHLCVVLDLYSKLIMGWSMHHRQDRPHGGSRGTDGGLAARGRRRGDPAFRSRRAVHQRYVPEVPRQPCLGLQHERGRP

SEQ ID NO: 28 - protein, 52 aa, Hepatitis X Related Virus
RCSRWMTTRATCIATQCRSPPSSTIRCESRPPCNMLSVYWFNRPLWAKTQLM

SEQ ID NO: 29 - protein, 67 aa, Hepatitis X Related Virus
PQGRRFFRPKGRLGGVRRGSPTLFRRSLSKEASSGSVFHTVSMVSITVSSPFSWRQTTRSRYLLSMM

SEQ ID NO: 30 - protein, 57 aa, Hepatitis X Related Virus
AQALRFGPGRAWAFERRKSRMRSIWSSLRPMMPARRICSSRASSPMGALKFRTARTM

SEQ ID NO: 31 - DNA, 20 bp, Artificial Sequence
  Sense Primer
cccgccccgctgatgaaaag

SEQ ID NO: 32 - DNA, 21 bp, Artificial Sequence
  Antisense Primer
gcgatggttgagtctcgacta

SEQ ID NO: 33 - DNA, 17 bp, Artificial Sequence
  Antisense Primer
aggtagcaggcgatatc

SEQ ID NO: 34 - DNA, 19 bp, Artificial Sequence
  Sense Primer
ccttctggagagggatttc

SEQ ID NO: 35 - DNA, 20 bp, Artificial Sequence
  Antisense Primer
tgttacctgctacttcgtgc

SEQ ID NO: 36 - DNA, 20 bp, Artificial Sequence
  Sense Primer
tagagttgcgaggcgtgacc

SEQ ID NO: 37 - DNA, 21 bp, Artificial Sequence
  Antisense Primer
ccttatccagtggcttttggc

SEQ ID NO: 38 - DNA, 21 bp, Hepatitis X Related Virus
tagtcgagactcaaccatcgc

SEQ ID NO: 39 - DNA, 20 bp, Artificial Sequence
  Antisense Primer
gcactgccgagttacatggc

(19) United States
(12) Patent Application Publication
(10) Pub. No.: US 2008/0102077 A1
(43) Pub. Date: May 1, 2008

(54) Nucleic and Protein Sequences from the Hxhv Virus and Uses Thereof

(75) Inventors: Isabelle Chemin, Caluire (FR); Christian Trepo, Bron (FR)

Correspondence Address: OLIFF & BERRIDGE, PLC, P.O. BOX 320850, ALEXANDRIA, VA, 22320-4850, US

(73) Assignee: Institut National de la Sante et de la Recherche Medicale (I.N.S.E.R.M.), Paris Cedex 13 (FR)

(21) Appl. No.: 10/563,134

(22) PCT Filed: Jul. 5, 2004
(86) PCT No.: PCT/FR04/50310

§ 371 Date: Mar. 8, 2007

(30) Foreign Application Priority Data
Jul. 4, 2003 (FR) 03 08174

(51) Int. Cl. A61K 31/70; A61K 38/00; A61K 39/00; A61P 43/00; C07H 21/04; C07K 16/00; C12N 1/16; C12N 1/20; C12N 15/00; C12N 5/00; C12N 5/02; C12N 5/04; C12P 21/04; C12Q 1/70

(52) U.S. Cl. 424/186.1; 424/93.21; 435/252.8; 435/255.1; 435/320.1; 435/325; 435/348; 435/358; 435/365; 435/367; 435/370; 435/372; 435/5; 435/69.1; 514/44; 530/300; 530/387.1; 530/387.9; 536/22.1; 536/23.1; 536/24.1; 536/24.3; 536/24.33

(57) ABSTRACT

The disclosure relates to isolated nucleic acid sequences obtainable from the genome of the HXHV virus, specifically, nucleic acid sequences comprising the sequence identified by SEQ ID NO: 4 or the complementary sequence to SEQ ID NO: 4, and to polypeptides coded by the nucleic acid sequences and uses thereof.

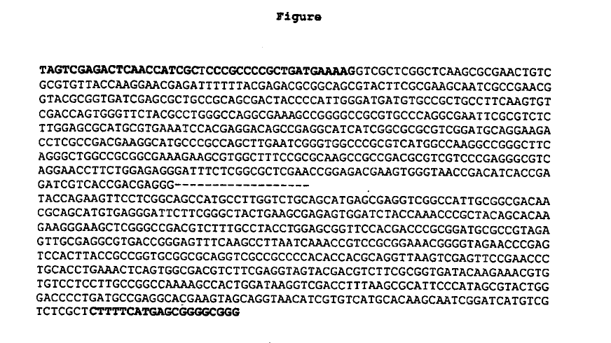

Hepatitis is the most important of the transmissible diseases. The method of transmission is most commonly transfusion, organ transplantation and hemodialysis, but hepatitis can also be transmitted by ingestion of contaminated food or water or by contact between individuals.

Viral hepatitis is induced by various viral agents that differ from one another by virtue of their genomes and their methods of replication. Viral hepatitis causes damage to the liver with varying degrees of severity. Close to a billion individuals in the world suffer from viral hepatitis. There are serious risks involved with chronic forms of hepatitis, which can progress to cirrhosis or hepatocarcinoma. Viral hepatitis can be diagnosed by the demonstration of well-defined symptoms, such as jaundice, high transaminase levels (aspartate transaminase or AST, alanine transaminase or ALT, lactate dehydrogenase or LDH), and hepatic lesions. However, despite knowledge of various viruses for hepatitis A, B, C, D, E, G and TTV, 5% of all incidences of hepatitis and 40% of instances of fulminant hepatitis remain unexplained, hence the hypothesis that unknown hepatitis viruses exist. These forms of hepatitis of unknown etiology are both post-transfusional and sporadic, chronic or fulminant. They are commonly called hepatitis X.

The hepatitis G (GBV-A, GBV-B, GBV-C) and TTV viruses recently identified do not appear to be pathogenic in humans and cannot therefore explain the cases of hepatitis of unknown etiology or hepatitis X.

Based on a case of severe hepatitis of unknown etiology, in a patient in whom treatment with interferon made it possible to normalize transaminases, a new virus called HXHV, associated with hepatitis X, has been described. The genome of the HXHV virus is an at least partially single-stranded DNA genome which comprises one or more reading frames encoding one or more protein(s) or polyprotein(s); the genome comprises a nucleotide sequence capable of hybridizing to the XH nucleotide sequence or to the nucleotide sequence complementary to the XH sequence. The XH sequence is represented in the sequence identifier of the present application as SEQ ID No. 1. The XH sequence is rich in GC (62%) and has four open reading frames (ORF1, ORF2, ORF3, ORF4). This isolated sequence has been characterized and no sequence homology with human genomic DNA and with the sequences present in the databases has been found. All the information concerning the HXHV virus is contained in patent application PCT/FR02/04578 filed under the Applicants' names.

The present inventors have now isolated and characterized a novel nucleotide sequence of the HXHV virus. This sequence, called XH1, is GC-rich (61.2%), which is comparable with the GC content of the previously isolated XH sequence. The XH1 sequence is referenced in the sequence identifier as SEQ ID No. 4. The XH1 sequence exhibits no significant homology or identity with any of the sequences available in the databases. It has 5 open reading frames. The DNA sequences corresponding to said open reading frames are respectively identified as SEQ ID Nos. 5 to 9 in the sequence identifiers. As is common practice in the field of virology, the present inventors have generated the complementary DNA strand of the XH1 sequence and have also investigated whether potential open reading frames exist on the complementary DNA strand. They have identified 8 open reading frames which are respectively represented as SEQ ID Nos. 10 to 17. The polypeptide sequences corresponding to said reading frames are respectively identified as SEQ ID Nos. 18 to 30 in the sequence identifier. The abovementioned sequences and the fragments thereof are used for the detection of the HXHV virus.

Thus, the present invention relates to:
a nucleic acid sequence that can be obtained from the HXHV virus genome, said nucleic acid sequence comprising or consisting of SEQ ID No. 4;
a nucleotide fragment of isolated DNA comprising or consisting of a DNA or RNA nucleotide sequence of at least 12 contiguous nucleotides, preferably of at least 15 or of at least 18 contiguous nucleotides, and advantageously of at least 20, 21, 22, 23, 24, 27, 30, 33, 36, 39, 42, 45, 48, 51 or 54 contiguous nucleotides, of the DNA nucleotide sequence SEQ ID No. 4 or of the DNA sequence complementary to SEQ ID No. 4; or of a nucleotide sequence which exhibits, over at least 12 contiguous nucleotides, preferably over at least 15 or at least 18 contiguous nucleotides, and advantageously over at least 20, 21, 22, 23, 24, 27, 30, 33, 36, 39, 42, 45, 48, 51 or 54 contiguous nucleotides, at least 90%, preferably at least 92% or 95% or at least 98% or 99% homology or identity with respect to the sequence represented in SEQ ID No. 4 or with respect to the DNA sequence complementary to SEQ ID No. 4; with the exclusion of the fragments that consist of one of the following nucleotide sequences: TAGTCGAGACTCAACCATCGC, CCCGCCCCGCTGATGAAAAG and of nucleotide sequences complementary to said sequences; or on the condition that, over 20 contiguous nucleotides or 21 contiguous nucleotides, said DNA nucleotide fragment does not exhibit 100% homology or identity with a nucleotide fragment of the sequence referenced SEQ ID No. 1 or of the sequence complementary to SEQ ID No. 1. Said fragment is in particular chosen from the fragments in which said contiguous nucleotides belong to one of the following segments: a segment whose sequence begins at nucleotide 2 and ends at nucleotide 286 of SEQ ID No. 4, a segment whose sequence begins at nucleotide 4 and ends at nucleotide 144 of SEQ ID No. 4, a segment whose sequence begins at nucleotide 180 and ends at nucleotide 1004 of SEQ ID No. 4, a segment whose sequence begins at nucleotide 614 and ends at nucleotide 820 of SEQ ID No. 4, a segment whose sequence begins at nucleotide 1228 and ends at nucleotide 1314 of SEQ ID No. 4, or the complementary fragments; a segment whose sequence begins at nucleotide 1283 and ends at nucleotide 1197 of the sequence complementary to SEQ ID No. 4, a segment whose sequence begins at nucleotide 1264 and ends at nucleotide 1067 of the sequence complementary to SEQ ID No. 4, a segment whose sequence begins at nucleotide 1209 and ends at nucleotide 1099 of the sequence complementary to SEQ ID No. 4, a segment whose sequence begins at nucleotide 819 and ends at nucleotide 736 of the sequence complementary to SEQ ID No. 4, a segment whose sequence begins at nucleotide 800 and ends at nucleotide 6 of the sequence complementary to SEQ ID No. 4, a segment whose sequence begins at nucleotide 784 and ends at nucleotide 629 of the sequence complementary to SEQ ID No. 4, a segment whose sequence begins at nucleotide 610 and ends at nucleotide 410 of the sequence complementary to SEQ ID No. 4, a segment whose sequence begins at nucleotide 391 and ends at nucleotide 221 of the sequence complementary to SEQ ID No. 4, or the complementary fragments; and preferably a fragment comprising or consisting of any one of the sequences SEQ ID Nos. 5 to 17 of any one of the DNA sequences complementary to SEQ ID Nos. 5 to 17 (the segment whose sequence begins at nucleotide 180 and ends at nucleotide 1004 of SEQ ID No. 4 encodes a transposase/integrase protein);
the product of transcription of the sequence comprising or consisting of SEQ ID No. 4 or the product of transcription of a fragment as defined above, or the product of the transcription of the sequence comprising or consisting of the sequence complementary to SEQ ID No. 4;
a DNA molecule which comprises or consists of a DNA nucleotide sequence represented in SEQ ID No. 4 or in that it comprises at least one DNA nucleotide fragment as defined above, or the sequences complementary thereto;
an RNA molecule which comprises or consists of an RNA nucleotide sequence which is the product of transcription of a DNA nucleotide sequence represented in SEQ ID No. 4 or of the sequence complementary to SEQ ID No. 4 or which is the product of transcription of at least one fragment as defined above, or the sequences complementary thereto.

The homology and identity above cover the functional equivalents of the sequence SEQ ID No. 4, i.e. the DNA sequences in which at least one codon can be replaced with another codon while at the same time encoding an identical amino acid. This is referred to as degeneracy of the genetic code. Thus, the codes for arginine, for serine and for leucine exhibit a degeneracy of the order of 6 (i.e. there are six different codons for each of them), whereas the codes for other amino acids, such as glutamic acid, glutamine, tyrosine, histidine and some others, exhibit a degeneracy of the order of 2. Of all the amino acids, only tryptophan and methionine have a degeneracy of the order of 1. It is therefore clear that, for the expression of a polypeptide whose sequence is represented in SEQ ID Nos. 18 to 30, it is possible to use variant and functional nucleic acid sequences whose codon compositions are different from the nucleic acid sequence represented in SEQ ID No. 4 or from the sequence complementary thereto.

The homology or identity defined above is also directed toward the variants of the HXHV virus and the mutant sequences of the HXHV virus, and in particular those derived from natural variability. In fact, it is well known to specialists that viruses have relatively high spontaneous and induced mutation rates.

The invention also relates to:
a polypeptide comprising a polypeptide sequence encoded by a sequence or by a fragment as defined above or by functional equivalents thereof or by a nucleotide sequence which exhibits at least 90% homology or identity, preferably at least 92% or 95% homology or identity, and advantageously at least 98% or 99% homology or identity, with respect to the sequence represented in SEQ ID No. 4 or with respect to the sequence complementary to SEQ ID No. 4, on the condition that the sequences TAGTCGAGACTCAACCATCGC, CCCGCCCCGCTGATGAAAAG, and the nucleotide sequences complementary to said sequences are excluded; or on the condition that, over 20 contiguous nucleotides or 21 contiguous nucleotides, the DNA nucleotide fragment does not exhibit 100% homology or identity with a nucleotide fragment of the sequence referenced SEQ ID No. 1 or of the sequence complementary to SEQ ID No. 1;
a polypeptide whose polypeptide sequence comprises or consists of any one of the sequences SEQ ID Nos. 18 to 30 or of a polypeptide sequence functionally equivalent to said sequences;
a polypeptide fragment, characterized in that it comprises or consists of a peptide sequence of at least 4 contiguous amino acids, preferably of at least 5, 6, 7, 8, 9, 10, 11, 12, 13, 14, 15, 16, 17 or 18 amino acids, of any one of the peptide sequences represented in SEQ ID Nos. 18 to 30 or of a peptide sequence functionally equivalent to said sequences SEQ ID Nos. 18 to 30; it being understood that the term “peptide sequence functionally equivalent” is intended to mean a peptide sequence which is recognized by antibodies directed against the HXHV virus;
a polypeptide fragment which comprises or which consists of a peptide sequence represented in any one of SEQ ID Nos. 18 to 30 or a peptide sequence functionally equivalent to any one of SEQ ID Nos. 18 to 30; it being understood that the term “peptide sequence functionally equivalent” is intended to mean a peptide sequence which is recognized by antibodies directed against the HXHV virus;
an epitope, characterized in that it comprises or consists of a peptide sequence of at least 6, 8, 9, 10, 12, 15 or 18 amino acids, and at most of 10, 12, 15 or 18 amino acids, in particular in that its sequence consists of a peptide sequence of 6 to 10 amino acids, of 6 to 12 amino acids, of 6 to 15 amino acids, of 6 to 18 amino acids, of 8 to 10 amino acids, of 8 to 12 amino acids, of 8 to 15 amino acids, of 8 to 18 amino acids and of 15 to 18 amino acids, of any one of the sequences represented in SEQ ID Nos. 18 to 30 or of a polypeptide sequence functionally equivalent to said sequences SEQ ID Nos. 18 to 30; it being understood that said epitope is recognized by antibodies directed against the HXHV virus.

The term “polypeptide” denotes a peptide, in the isolated state, having a series of a variable number of amino acids, such as an oligopeptide, a protein, a fusion protein, a fusion peptide or a synthetic peptide. A polypeptide can be obtained by various techniques well known to those skilled in the art, and in particular by chemical synthesis or genetic recombination techniques. The polypeptides according to the invention can be obtained by conventional synthesis methods, for example with an automatic peptide synthesizer, or by genetic engineering techniques comprising the insertion of a DNA sequence encoding said polypeptide into an expression vector such as a plasmid or a virus, and the transformation of cells with this expression vector and culturing of these cells.

The expression “peptide sequence functionally equivalent to a reference peptide sequence” is intended to mean an amino acid sequence modified by insertion and/or deletion and/or substitution and/or extension and/or shortening and/or chemical modification of one or more amino acids, provided that these modifications substantially preserve, or even develop, the immunoreactive properties of said reference peptide sequence.

Thus, the term “functionally equivalent sequences” is intended to mean sequences which conserve the immunoreactive properties of SEQ ID Nos. 18 to 30 or of fragments thereof, in particular the sequences in which one or more amino acid(s) is or are substituted with one or more other amino acids; the sequences in which one or more amino acids of the L series is replaced with an amino acid of the D series, and vice versa; the sequences into which an amino acid side chain modification has been introduced, such as an amine function acetylation, a thiol function carboxylation or a carboxyl function esterification; a modification of the peptide bonds, such as, for example, carba, retro, inverso, retro-inverso, reduced and methyleneoxy bonds.

For example, one or more amino acid(s) in the sequences of the polypeptides of the invention can be substituted with one or more other amino acid(s) of similar polarity which act as functional equivalents. Substitutions for an amino acid in polypeptide sequences of interest can be determined from other members of the class to which the amino acid belongs. For example, nonpolar (hydrophobic) amino acids comprise alanine, leucine, isoleucine, valine, proline, phenylalanine, tryptophan and methionine. Polar neutral amino acids comprise glycine, serine, threonine, cysteine, tyrosine, asparagine and glutamine. Positively charged (basic) amino acids comprise arginine, lysine and histidine. Negatively charged (acidic) amino acids comprise aspartic acid and glutamic acid. Other substitutions for an amino acid in polypeptide sequences of interest can be determined from the information contained in the article by Kramer A. et al. (Molecular Immunology, Vol. 32, No. 7, pp. 459-465 (1995)). These authors constituted libraries in which, in order to reduce the problem of combinatorial explosion of the number of molecules, they used groups of amino acids consisting of amino acids having similar physicochemical properties, and they are the amino acids grouped together into each of these six grouped, listed below, which are considered mainly to be equivalent in the present invention.

Group 1: alanine, proline, glycine.

Group 2: aspartic acid, glutamic acid.

Group 3: histidine, lysine, arginine.

Group 4: asparagine, glutamine, serine, threonine.

Group 5: phenylalanine, tyrosine, tryptophan.

Group 6: isoleucine, leucine, valine, methionine.

The equivalence of a peptide sequence relative to a reference peptide sequence can be defined by its identity or its homology, expressed as a percentage, with said reference sequence. This percentage is determined, for a series of a given number of contiguous amino acids, by alignment of the two sequences, displacement of one with respect to the other, and comparison of the amino acids in the two sequences. The percentage identity is determined from the number of amino acids which are identical to amino acids of the reference sequence, in the same position. The percentage homology is determined from the number of amino acids which are equivalent to amino acids of the reference sequence, in the same position.

The invention also relates to an expression cassette that is functional in a cell derived from a prokaryotic or eukaryotic organism allowing the expression of a nucleic acid sequence or of a DNA fragment or of a DNA molecule as described above, placed under the control of the elements required for its expression. The expression cassette is characterized in that it is functional in a cell derived from a prokaryotic organism, in particular E. coli, or from a eukaryotic organism, in particular cells originating from animals such as mammals, reptiles or insects, in particular COS, CHO, BHK, PK 15 and RK 13 cells; human osteosarcoma cell lines (143 B cells), HeLa human cell lines and human hepatoma cell lines (of the HepG2 type); insect cell lines (for example from Spodoptera frugiperda); or a lower eukaroytic organism, in particular cells from yeast, such as Saccharomyces, Schizosaccharomyces, Kluveromyces, Hanseluna, Yarowia, Schwaniomyces, Zygosaccharomyces and Pichia, and preferably chosen from Saccharomyces cerevisiae, Saccharomyces carlsbergensis, Schizosaccharomyces pombe, Kluveromyces lactis and Pichia pastoris cells.

The invention also relates to a vector comprising said expression cassette; to a cell derived from a prokaryotic, eukaryotic or lower eukaryotic organism, preferably a eukaryotic or lower eukaryotic organism as defined above or a vector as defined above; and to the polypeptide that can be produced by the expression cassette, the vector or the cell.

A subject of the invention is a method for preparing a polypeptide or a polypeptide fragment as defined above, which consists in culturing a host cell corresponding to the definitions above in an appropriate culture medium, said host cell being transformed with an expression vector which contains a DNA nucleic acid sequence as defined above or a DNA nucleotide fragment as defined above or a DNA molecule as defined above, and in purifying said polypeptide produced, to a required degree of purity.

A subject of the invention is also an immunogenic polypeptide, said polypeptide comprising or consisting of a polypeptide or peptide sequence as defined above. Such an immunogenic polypeptide is used for the production of monoclonal or polyclonal antibodies or of fragments of said antibodies, and the invention encompasses the monoclonal or polyclonal antibodies or fragments thereof, that are obtained by immunization of a mammalian animal (rabbit, rat, mouse) with such an immunogenic peptide.

The production of monoclonal or polyclonal antibodies is well known to those skilled in the art. By way of reference, mention may be made of Köhler G. and Milstein C. (1975): Continuous culture of fused cells secreting antibody of predefined specificity, Nature 256: 495-497 and Galfre G. et al. (1977): Nature, 266: 522-550, for the production of monoclonal antibodies, and Roda A., Bolelli G. F.: Production of high-titer antibody to bile acids, Journal of Steroid Biochemistry, Vol. 13, pp. 449-454 (1980), for the production of polyclonal antibodies. Antibodies can also be produced by immunization of mice, rats or rabbits with the HXHV viral particles. For the production of polyclonal and monoclonal antibodies, the immunogen can be coupled to serum albumin (SA peptide) or to Keyhole Limpet Hemocyanine (KLH peptide) as an immunization carrier. The antibodies are subsequently screened for their specificity using the usual techniques, such as ELISA or Western blotting assays. For the production of monoclonal antibodies, the animals are given an injection of immunogen using Freund's complete adjuvant. The sera and the hybridoma culture supernatants derived from the immunized animals are analyzed for their specificity and their selectivity using conventional techniques, such as, for example, ELISA or Western blotting assays. The hybridomas producing the most specific and the most sensitive antibodies are selected. Monoclonal antibodies can also be produced in vitro, by cell culture of the hybridomas produced or by recovery of ascites fluid, after intraperitoneal injection of the hybridomas into mice. Whatever the method of production, in supernatant or in ascites, the antibodies are subsequently purified. The purification methods used are essentially filtration on ion exchange gel and exclusion chromatography or affinity chromatography (protein A or G). A sufficient number of antibodies are screened in functional assays so as to identify the most effective antibodies. The in vitro production of antibodies, of antibody fragments or of antibody derivatives, such as chimeric antibodies produced by genetic engineering, is well known to those skilled in the art. It is advantageous to use humanized antibodies. “Humanized” forms of nonhuman antibodies, for example murine antibodies, are chimeric antibodies which comprise a minimal sequence derived from a nonhuman immunoglobulin. Most humanized antibodies are human immunoglobulins (recipient antibody) in which residues of a hypervariable region of the recipient are replaced with residues of a hypervariable region of a nonhuman donor species (donor antibody), such as mouse, rat, rabbit or nonhuman primate, having the desired specificity, affinity and capacity. In certain cases, the residues (FR) of the Fv region of the human immunoglobulin are replaced with corresponding nonhuman residues. Furthermore, the humanized antibodies can comprise residues which are not found in the recipient antibody or in the donor antibody. These modifications are carried out in order to improve the performance levels of the antibody. In general, the humanized antibody will comprise at least and preferably two variable domains, in which all or virtually all of the hypervariable loops correspond to a nonhuman immunoglobulin and all or virtually all of the FR regions will be those of a human immunoglobulin. The humanized antibodies may optionally also comprise at least one part of a constant (Fc) region of an immunoglobulin, such as a human immunoglobulin (Jones et al., Nature 321: 522-525 (1986); Reichmann et al., Nature 332: 323-329 (1988); and Presta et al., Curr. Op. Struct. Biol. 2: 593-596 (1992)).

More particularly, the term “antibody fragment” is intended to mean the F(ab)2, Fab, Fab′ or sFv fragments (Blazar et al., 1997, Journal of Immunology 159: 5821-5833 and Bird et al., 1988, Science 242: 423-426) of a natural antibody, and the term “derivative” is intended to mean, inter alia, a chimeric derivative of a natural antibody (see, for example, Arakawa et al., 1996, J. Biochem 120: 657-662 and Chaudray et al., 1989, Nature 339: 394-397). These antibody fragments and antibody derivatives conserve the ability to selectively bind to the target antigen.

The monoclonal or polyclonal antibody thus obtained, or fragment thereof, is incorporated into a diagnostic composition which is used in a method for detecting at least one polypeptide or one peptide fragment as defined above in a biological sample, according to which the biological sample is brought into contact with the composition under predetermined conditions which allow the formation of antibody/antigen complexes, and the formation of said complexes is detected.

A subject of the invention is also a diagnostic composition which comprises a polypeptide or a peptide fragment as defined above, and a method for detecting antibodies directed against the HXHV virus or at least against a polypeptide or a peptide fragment of the invention, according to which a biological sample suspected of being or of possibly having been infected with the HXHV virus is brought into contact with the diagnostic composition under predetermined conditions which allow the formation of antibody/antigen complexes, and the formation of said complexes is detected. This is because it is known that, during infection with a viral agent, the host develops antibodies directed against this viral agent (humoral response).

A subject of the present invention is also the biological material for preparing a pharmaceutical composition for use in the treatment of human beings or of animals infected with at least the HXHV virus and the immunogenic or vaccine compositions which can be used to produce therapeutic vaccines against an HXHV virus infection and prophylactic vaccines for preventing a potential HXHV virus infection, said immunogenic preparations comprising at least one natural, recombinant or synthetic polypeptide or peptide fragment of the invention, combined with a pharmaceutically acceptable vehicle and/or adjuvant and/or diluent and/or excipient.

A subject of the present invention is also the use of at least one monoclonal or polyclonal antibody or of at least one fragment of said antibodies of the invention, specific for at least one polypeptide or one peptide fragment as defined above, for preparing a pharmaceutical composition which, when administered to a patient infected with the HXHV virus, has the ability to reduce, or even inhibit, the proliferation and/or the replication of the virus. These antibodies or fragments thereof are called neutralizing antibodies.

The term “biological sample” is intended to mean, for example, blood, serum, plasma or tissue samples, such as liver biopsy extracts.

The vaccines prepared are injectable, i.e. in liquid solution or in suspension. As an option, the preparation can also be emulsified. The antigenic molecule can be mixed with excipients which are pharmaceutically acceptable and compatible with the active ingredient. Examples of favorable excipients are water, a saline solution, dextrose, glycerol, ethanol or equivalents and combinations thereof. If desired, the vaccine can contain minor amounts of auxiliary substances, such as wetting agents or emulsifiers, pH-buffering agents or adjuvants such as aluminum hydroxide, muramyl dipeptide, or variations thereof. In the case of the peptides, the coupling thereof to a larger molecule (KLH, tetanus toxin) sometimes increases the immunogenicity. The vaccines are administered conventionally by injection, for example intramuscular injection. Additional formulations that are favorable with other methods of administration include suppositories and sometimes oral formulations.

The term “pharmaceutically acceptable vehicle” is intended to mean the carriers and vehicles that can be administered to human beings or to an animal, as described, for example, in Remington's Pharmaceutical Sciences 16^{th} ed., Mack Publishing Co. The pharmaceutically acceptable vehicle is preferably isotonic or hypotonic or exhibits a weak hypertonicity and has a relatively low ionic strength. The definitions of the pharmaceutically acceptable excipients and adjuvants are also given in Remington's Pharmaceutical Sciences mentioned above.

A subject of the invention is also:
a probe, characterized in that it is capable of hybridizing, under given stringency conditions, to a nucleic acid sequence or to a nucleotide fragment of DNA or of RNA or to a DNA or RNA molecule of the invention; preferably, a probe of the invention comprises at least 12 nucleotides, preferably at least 17, 18, 19, 20, 21, 22, 23, 24 or 25 nucleotides and the hybridization is carried out under stringency conditions corresponding to a combination of temperature and salt concentration chosen approximately between 12 and 20° C. below the Tm (melting temperature) of the complex of probe/nucleotide sequence to be detected;
a primer, characterized in that it is capable of hybridizing, under given stringency conditions, to a nucleic acid sequence or to a nucleotide fragment of DNA or of RNA or to a DNA or RNA molecule of the invention; preferably, a primer of the invention comprises at least 12 nucleotides, preferably at least 17, 18, 19, 20, 21, 22, 23, 24 or 25 nucleotides and the hybridization is carried out under stringency conditions corresponding to a combination of temperature and salt concentration chosen approximately between 12 and 20° C. below the Tm (melting temperature) of the complex of primer/nucleotide sequence to be amplified and/or detected. The primers represented in SEQ ID Nos. 32 to 37 are novel and, as described in the experimental section, pairs of primers are used for amplifying the HXHV virus nucleic acids, said pairs of primers being chosen preferably from the following pairs: SEQ ID No. 31/SEQ ID No. 32, SEQ ID No. 31/SEQ ID No. 33, SEQ ID No. 34/SEQ ID No. 35, and SEQ ID No. 36/SEQ ID No. 37;
an anti-nucleic acid antibody, characterized in that it is capable of binding to a nucleic acid sequence or to a nucleotide fragment of DNA or of RNA or to a DNA or RNA molecule;
a diagnostic composition, characterized in that it comprises at least one probe or one primer or one anti-nucleic acid antibody as defined above;
a method for detecting viral DNA and/or RNA, according to which a biological sample is taken from a patient suspected of being or of possibly having been infected with the HXHV virus, said sample is, if necessary, treated so as to extract the DNA and/or the RNA therefrom, said sample is brought into contact with at least one probe or one primer of the invention, under given stringency conditions, and the presence of viral DNA and/or RNA in the sample is detected either by demonstrating hybridization of said viral DNA and/or RNA with at least one probe, or by amplifying said DNA and/or RNA (for example, as described in the experimental section of the invention); and
a method for detecting viral DNA and/or RNA, according to which a serum or plasma sample is taken from a patient, said sample is, if necessary, treated so as to extract the DNA and/or RNA therefrom, said sample is brought into contact with at least one anti-nucleic acid antibody, said antibody being optionally labeled with any appropriate label, and the formation of a nucleic acid/antibody complex is demonstrated.

The production of polynucleotides, probes or primers is part of the general knowledge of those skilled in the art. Mention may in particular be made of the use of restriction enzymes, and chemical synthesis on an automatic synthesizer. The probes and primers capable of hybridizing, under given stringency conditions, to a DNA or RNA nucleotide sequence or to a nucleotide fragment as defined above are part of this definition. It is within the scope of those skilled in the art to define the appropriate stringency conditions. Characteristic stringency conditions are those which correspond to a combination of temperature and salt concentration chosen approximately between 12 and 20° C. below the Tm (melting temperature) of the hybrid being studied. Reference may thus be made to the work by George H. Keller and Mark M. Manak, DNA PROBES, second edition, Stockton Press, 1993, 49 West 24^{th} St., New York, N.Y. 10010 USA. The stringency conditions for discriminating even a single point mutation in a nucleic sequence have been known at least since 1979. By way of examples, mention may be made of Wallace R. B et al., DNA. Nucleic Acids Res. 6, 3543-3557 (1979), Wallace R. B. et al., Science, 209, 1396-1400 (1980), Itakura K. and Riggs A. D., Science, 209, 1401-1405 (1980), Suggs S. V. et al., PNAS, 78, 6613-6617 (1981), Wallace R. B. et al., DNA. Nucleic Acids Res., 9, 3647-3656 (1981), Wallace R. B. et al. DNA. Nucleic Acids Res., 9, 879-894 (1981) and Conner B. J. et al., PNAS, 80, 278-282 (1983). Moreover, techniques for the production of anti-nucleic acid antibodies are known. By way of examples, mention may be made of Philippe Cros et al., Nucleic Acides Researc, 1994, Vol. 22, No. 15, 2951-2957; Anderson, W. F. et al. (1988) Bioessays, 8 (2), 69-74; Lee, J. S. et al. (1984) FEBS Lett., 168, 303-306; Malfoy, B. et al. (1982) Biochemistry, 21(22), 5463-5467; Stollar, B. D. et al., J. J. (eds) Methods in Enzymology, Academic Press, pp 70-85; Traincard, F. et al. (1989) J. Immunol. Meth., 123, 83-91 and Traincard, F. et al. (1989) Mol. Cell. Probes, 3, 27-38).

The invention also relates to:
a vaccine composition comprising a DNA sequence encoding at least one polypeptide or one peptide fragment of the invention, said DNA being mixed with an appropriate and pharmaceutically acceptable vehicle and/or diluent and/or excipient;
an antisense or anti-gene oligonucleotide, characterized in that it is capable of interfering specifically with the synthesis of at least one polypeptide or one peptide fragment of the invention;
a pharmaceutical composition, characterized in that it comprises at least one antisense oligonucleotide or one anti-gene oligonucleotide;
a vector, characterized in that it comprises at least one gene of therapeutic or vaccine interest, said gene encoding in particular
(i) either at least a polypeptide or a peptide fragment of the invention;
(ii) or at least all or part of an antibody capable of binding to at least one polypeptide or peptide fragment defined in (i);
(iii) or at least a molecule that inhibits at least one polypeptide or peptide fragment defined in (i);
(iv) or at least a ligand or any part of a ligand capable of binding to at least one polypeptide or peptide fragment defined in (i) and/or of inhibiting its function;
a therapeutic or vaccine composition, characterized in that it comprises, inter alia, a vector as defined above and in that said gene of interest is placed under the control of elements that ensure its expression in vivo;
a biological material for the preparation of a pharmaceutical or vaccine composition, comprising at least one cell, in particular a cell that does not naturally produce antibodies, in a form that allows it to be administered to a human or animal mammalian organism and also optionally to be cultured beforehand, said cell being genetically modified in vitro with at least one nucleic acid sequence or with at least one nucleotide fragment or with at least one DNA molecule or with at least one vector of the invention, said nucleic acid sequence, nucleotide fragment, DNA molecule and gene of the vector encoding, in vivo, at least one polypeptide or one peptide fragment of the invention or encoding at least all or part of an antibody which is capable of binding to a polypeptide or peptide fragment of the invention or encoding at least one molecule that inhibits the function and/or the binding and/or the expression of at least one polypeptide or of a peptide fragment;
a therapeutic or vaccine composition comprising said biological material;
a genetically modified cell, in particular chosen from eukaryotic cells, such as COS, CHO, Vero, BHK, PK 15 and RK 13 cells; human osteosarcoma cell lines, HeLa human cell lines and human hepatoma cell lines, insect cell lines; cells of lower eukaryotes, such as yeast cells, in particular cells derived from Saccharomyces, Schizosaccharomyces, Kluveromyces, Hanseluna, Yarowia, Schwaniomyces, Zygosaccharomyces and Pichia, and preferably chosen from Saccharomyces cerevisiae, Saccharomyces carlsbergensis, Schizosaccharomyces pombe, Kluveromyces lactis and Pichia pastoris cells; prokaryotic cells, such as those derived from E. coli; said cells being transformed with at least one nucleic acid sequence or with at least one nucleotide fragment or with a DNA molecule or with a vector of the invention; and
a pharmaceutical or vaccine composition comprising such a cell.

The pharmaceutical compositions defined above are DNA vaccine compositions that are particularly advantageous, in particular with respect to the “conventional” vaccine compositions based on recombinant protein. This is because the use for vaccine purposes of recombinant proteins is a laborious and expensive system, in particular because it requires very substantial recombinant antigen purification steps. Furthermore, one of the difficulties encountered is that of obtaining a vaccine persistence that is sufficiently long to maintain a good immune memory. Conversely, the method of immunization with DNA, the advantages of which are inherent in the intrinsic properties of the DNA, is simple and relatively inexpensive and is carried out simply by intramuscular or intradermal injection. Furthermore, it should be noted that:
DNA vaccines are noninfectious/nonreplicative,
because immunization with DNA is a form of in vivo transfection, the viral antigen is expressed in the mammalian cells in its natural conformation,
as in the case of a viral infection, a broad immune response, both humoral and cellular, is induced, and
furthermore, DNA vaccines can be readily combined because of their physicochemical homogeneity.

Finally, a subject of the invention is a method for evaluating a therapeutic agent, according to which given doses, as one dose or as repeated doses at given time intervals, of at least one polypeptide or one peptide fragment of the invention, that is natural, recombinant or synthetic, or else obtained from a biological sample optionally after prior treatment of said biological sample infected with the HXHV virus, are administered to an animal, a biological sample is taken from the animal, preferably blood or serum, and the following are carried out:
(i) an assay of an antibody or antibodies specific for the polypeptide or for the polypeptide fragment; and/or
(ii) an assay of the cellular immune response induced against the polypeptide or the polypeptide fragment, for example by means of a test for in vitro activation of “helper” T lymphocyte cells specific for the polypeptide or for the polypeptide fragment.

### FIGURE

The FIGURE represents the partial sequencing of the band of approximately 1.3 Kb. In the FIGURE, the position of the unsequenced fragment of approximately 200 base pairs is represented by the symbols (−). In the FIGURE, the nucleotide fragments indicated in bold correspond to nucleotide fragments that exhibit 100% sequence homology or identity with SEQ ID No. 1. Their respective positions relative to SEQ ID No. 1 are as follows: 253-233, 254-273, 273-254.

### EXAMPLES

### Example 1

### Extraction and Extension

The nucleic acids were extracted from 140 μl of a serum sample from a patient characterized as being HXHV positive by PCR (polymerase chain reaction) amplifications, as described in patent application PCT/FR02/04578, using the QIAamp Viral mini spin Kit (trade name) from the company Qiagen, according to the protocol recommended by the supplier.

A biotinylated primer (Comp S6M13-biotin), the sequence of which is represented below, was subsequently used to extend the sequence SEQ ID No. 1 of interest. The antisense biotinylated primer used corresponds to the nucleotides 494-475 of SEQ ID No. 1.

| | |
|---|---|
| Antisense primer Comp S6M13: | |
| 5′-GCACTGCCGAGTTACATGGC-3′ | (SEQ ID No.) |

For the extension, the GENEAmp XL PCR Kit (trade name) from the company Roche was used, following the protocol recommended by the supplier.

The composition of the reaction mixture of 50 μl is as follows:

| | | |
|---|---|---|
| 25 mM Mg (OAC)₂ | 2.4 | μl |
| 2.5 mM of each dNTP | 4.0 | μl |
| primer Comp S6M13 | 2.0 | μl (20 picomole) |
| 3.3 × XL Buffer II | 15.1 | μl |
| rTth DNA polymerase (2 U/μl) | 0.5 | μl (1 U) |
| DNA template | 10 | μl |
| Distilled water | 16 | μl |

The extension was carried out according to the following program:

The reaction mixture was heated at 92° C. for 2 minutes and subsequently subjected to 35 cycles, each cycle comprising heating at 92° C. for 30 seconds, heating at 55° C. for 30 seconds and heating at 68° C. for 3 minutes. The final extension was carried out by heating at 68° C. for 10 minutes, followed by cooling to 4° C.

### Example 2

### Capture of the Extended Double-Stranded DNA

The extension product obtained according to the protocol described in Example 1 was isolated using the Dynabeads Kilobase BINDER kit (trade name) from the company Dynal, according to the supplier's instructions. The beads (5 μl) were first washed twice in the binding buffer and resuspended in 20 μl of this buffer. A 20 μl aliquot of the extension product was added and incubated at ambient temperature for 3 hours on a roller so as to keep the beads in suspension. The double-stranded DNA was purified by two washes with a washing buffer and one wash with distilled water, and the beads were subsequently resuspended in 20 μl of distilled water and conserved at 4° C.

### Example 3

### Digestion and Circularization

5 μl of the double-stranded DNA, captured according to Example 2, were digested with the BsaWI enzyme (NEB), whose cleavage site corresponded to position 299 of SEQ ID No. 1, by heating at 60° C. for 2 hours. The enzyme was subsequently inactivated by heating at 80° C. for 20 minutes. After this, the tube was cooled slowly and the digested DNA was purified using the QIA quick PCR purification Kit (trade name) from the company Qiagen. The purified DNA was subsequently subjected to ligation at 4° C. overnight using the T4 ligase sold by the company Roche, and the ligation was finished off by heating at 65° C. for 10 minutes.

### Example 4

### Amplification

10 μl of the ligation product obtained according to Example 3 were used as a template for carrying out a semi-nested PCR using the GeneAmp XL PCR Kit (trade name) from the company Roche. The two rounds of PCR were carried out in the same way, according to the following protocol:

The reaction mixture was heated at 94° C. for 2 minutes and subsequently subjected to 35 cycles, each cycle comprising heating at 94° C. for 30 seconds, heating at 47° C. for 30 seconds and heating at 68° C. for 3 minutes. The reaction mixture was subsequently subjected to heating at 68° C. for 10 minutes, followed by cooling to 4° C.

First round of PCR:

Composition of the reaction mixture (50 μl):

| | | |
|---|---|---|
| 25 mM Mg(OAC)₂ | 2.4 | μl |
| 2.5 mM of each dNTP | 4.0 | μl |
| Sense primer 1M13 (25 μM) | 1.0 | μl |
| Antisense primer CIRC 1 (25 μM) | 1.0 | μl |
| 3.3 × XL Buffer II | 15.1 | μl |
| rTth DNA polymerase (2 U/μl) | 0.5 | μl (1 U) |
| DNA template | 10 | μl |
| Water | 16 | μl |

The following pairs of primers were used:

| | |
|---|---|
| Sense primer (1M13): | |
| 5′-CCCGCCCCGCTGATGAAAAG-3′ | (SEQ ID No. 31) |
| Antisense primer (CIRC 1) | |
| 5′-GCGATGGTTGAGTCTCGACTA-3′. | (SEQ ID No. 32) |

Second round of PCR:

Composition of the reaction mixture (50 μl):

| | | |
|---|---|---|
| 25 mM Mg(OAC)₂ | 2.4 | μl |
| 2.5 mM of each dNTP | 4.0 | μl |
| Sense primer 1M13 (25 μM) | 1.0 | μl |
| Antisense primer 6BRACE5′ (25 μM) | 1.0 | μl |
| 3.3 × XL Buffer II | 15.1 | μl |
| rTth DNA polymerase (2 U/μl) | 0.5 | μl (1 U) |
| Product of the 1^{st} round | 10 | μl |
| Water | 16 | μl |

The following pairs of primers were used:

| | |
|---|---|
| Sense primer (1M13): | |
| 5′-CCCGCCCCGCTGATGAAAAG-3′ | (SEQ ID No. 31) |
| Antisense primer (6BRACE5′): | |
| 5′-AGGTAGCAGGCGATATC-3′ | (SEQ ID No. 33) |

The locations of the primers in the XH sequence (SEQ ID No. 1) are respectively as follows:

| | |
|---|---|
| 1M13: | 254-273 |
| CIR 1: | 253-233 |
| 6BRACE5′: | 94-77 |

### Example 5

### Agarose Gel Electrophoresis and Hybridization

The amplification products obtained according to Example 4 were separated by 1.5% agarose gel electrophoresis. Three bands, the sizes of which were between 1.2 Kb and 2.5 Kb, were observed on the gel.

The amplified products were transferred onto a Hybond-N⁺ (trade name) nylon membrane (Amersham Biosciences UK Limited). The membrane was hybridized at 42° C. overnight with the complete XH fragment labeled at its 3′ end with ³²P (generated using the Ready to Go DNA Labelling beads kit (trade name) from the company Amersham Pharmacia Biotech Inc.). The following washes were carried out at 65° C.: 233 SSC, 15 minutes, twice; 133 SSC, 15 minutes, twice; 0.533 SSC, 15 minutes, twice. The membrane was subjected to autoradiography at −80° C. overnight. The three bands exhibited weak signals on the X-ray film after development.

### Example 6

### Cloning and Sequencing

The three bands were respectively cloned into the vector PCR2.1-TOPO (Invitrogen). The clones were subsequently screened by colony hybridization and identified using the EcoRI enzyme (Gibco BRL). The positive clones were selected so as to be sequenced.

The results of the sequencing revealed a 1133 base pair fragment. The search carried out in the database libraries showed no significant sequence homology. The 1133 base pair fragment is referenced in the sequence identifier as SEQ ID Nos. 2 and 3. It is also represented in the FIGURE.

### Example 7

### Repetition

Using the same digestion and circularization product described in Example 3, a further amplification was carried out according to the protocol described in Example 4, followed by agarose gel electrophoresis and the hybridization procedure described in Example 5. In this assay, a single band, the size of which was approximately 1.3 Kb, was observed on the gel. After cloning and sequencing, as described in Example 6, a 1133 base pair fragment corresponding to the fragment described in Example 6 (SEQ ID Nos. 2 and 3 and FIGURE) was obtained.

The relevance of this 1133 base pair fragment with respect to the HXHV virus was verified as described below.

Due to the limitations inherent in the sequencing used, the sequence of the band of approximately 1.3 Kb visualized on the gel proved to be incomplete. In fact, a fragment of approximately 1300 base pairs was expected. Thus, the present inventors therefore carried out, with a further procedure, a complete sequencing of the band of approximately 1.3 Kb, as described below. The part not sequenced in the initial sequencing, which corresponds to a fragment of approximately 200 base pairs, is represented, in terms of its location, in the FIGURE by the symbols (−). The first sequenced fragment is represented in SEQ ID No. 2 and the second sequenced fragment is represented in SEQ ID No. 3 in the sequence identifier.

### Example 8

### Relevance of the 1133 Base Pair Fragment

To verify the relevance of the 1133 base pair fragment with respect to the HXHV virus, nested PCRs were carried out in parallel.
Using fractions obtained on a sucrose gradient of 17 sera, 10 of which were positive for ORF4 of the HXHV virus described in patent application PCT/FR02/04578 and 7 of which were negative for this same ORF4, the nucleic acids were extracted and nested PCRs were carried out according to the following protocol using the Taq DNA polymerase from the company Promega:

The reaction mixture was heated at 94° C. for 5 minutes and subsequently subjected to 35 cycles, each cycle comprising heating at 94° C. for 45 seconds, heating at 43° C. for 45 seconds and heating at 72° C. for 1 minute. The reaction mixture was subsequently subjected to heating at 72° C. for 10 minutes, followed by cooling to 4° C.

First round of PCR:

Composition of the reaction mixture (50 μl)0:

| | | |
|---|---|---|
| Taq buffer with 10X MgCl₂ | 5.0 | μl |
| 10 mM of each dNTPs | 2.0 | μl |
| Sense primer XF4 (25 μM) | 1.0 | μl |
| Antisense primer XB12 (25 μM) | 1.0 | μl |
| Taq DNA polymerase (5 U/μl) | 0.5 | μl |
| DNA template | 10 | μl |
| Water | 30.5 | μl |

The following pairs of primers were used:

| | |
|---|---|
| Sense primer (XF4): | |
| 5′ CCTTCTGGAGAGGGATTTC 3′ | (SEQ ID No. 34) |
| Antisense primer (XB12): | |
| 5′ TGTTACCTGCTACTTCGTGC 3′ | (SEQ ID No. 35) |

Second round of PCR:

Composition of the reaction mixture (50 μl):

| | | |
|---|---|---|
| Taq buffer with 10X MgCl₂ | 5.0 | μl |
| 10 mM of each dNTPs | 2.0 | μl |
| Sense primer XF1 (25 μM) | 1.0 | μl |
| Antisense primer XB1 (25 μM) | 1.0 | μl |
| Taq DNA polymerase (5 U/μl) | 0.5 | μl |
| Product of the 1^{st} round | 10 | μl |
| Water | 35.5 | μl |

The following pairs of primers were used:

| | |
|---|---|
| Sense primer (XF1): | |
| 5′ TAGAGTTGCGAGGCGTGACC 3′ | (SEQ ID No. 36) |
| Antisense primer (XB1): | |
| 5′ CCTTATCCAGTGGCTTTTGGC 3′ | (SEQ ID No. 37) |

The locations of the primers in the sequence SEQ ID No. 4 are respectively as follows:

| | |
|---|---|
| XF4: | 482-500 |
| XB12: | 1255-1236 |
| XF1: | 944-963 |
| XB1: | 1186-1166 |

The amplification products obtained according to Example 4 were separated by 1.5% agarose gel electrophoresis. The amplified products were transferred onto a Hybond-N⁺ (trade name) nylon membrane (Amersham Biosciences UK Limited). The membrane was hybridized at 42° C. overnight with the product of the second round of PCR, labeled at its 3′ end with ³²P. The product of round 2 was purified with the Qiaquick Gel Extraction Kit (trade name) and labeled using the Ready to Go DNA Labelling beads kit (trade name) from the company Amersham Pharmacia Biotech Inc. The following washes were carried out at 65° C.: 233 SSC, 15 minutes, twice; 133 SSC, 15 minutes, twice; 0.533 SSC, 15 minutes, twice. The membrance was subjected to autoradiography at −80° C. overnight.

The band of expected size of approximately 240 base pairs is found in the amplified nucleic acids of 3 fractions out of the 10 that were positive for ORF4 of the HXHV virus. No band was revealed for the 7 fractions that were negative for ORF4 of the HXHV virus.
The nucleic acids extracted from 15 sera from non-A-E patients, 9 of which were positive for HXHV ORF4 and 6 of which were negative for this same ORF, were amplified by nested PCR with the same protocol as that described above. The amplified products obtained were subsequently separated by agarose gel electrophoresis, the membranes were hybridized and the bands were revealed by autoradiography according to the same protocol as that described above.

The band of expected size of approximately 240 base pairs is found in the nucleic acids amplified from 3 sera out of the 9 that were positive for ORF4 of the HXHV virus. No band was revealed for the 6 sera that were negative for ORF4 of the HXHV virus.

The results obtained from serum fractions and from sera therefore confirm that the 1133 base pair sequence is associated with the HXHV virus.

### Example 9

### Complete Sequencing of the Band of Approximately 1.3 Kb

The PCR products were purified by enzymatic digestion (Enzyme Exosup—trade name). The nucleic acids were quantified by fluorometric assay. The sequencing reaction was carried out by means of an enzymatic reaction in the presence of a primer specific for the region to be sequenced. The products were subsequently injected into the Applied Biosystem 3730 XL sequencer (trade name). The DNA sequence obtained is a sequence of 1314 base pairs represented in SEQ ID No. 4.

1. An isolated nucleic acid sequence that can be obtained from the HXHV virus genome, said nucleic acid sequence comprising the sequence SEQ ID NO: 4 or the sequence complementary to SEQ ID NO: 4.

2. An isolated nucleic acid sequence as claimed in claim 1, said nucleic acid sequence consisting of the sequence SEQ ID NO: 4 or of the sequence complementary to SEQ ID NO: 4.

3. A DNA nucleotide fragment, comprising:
a nucleotide sequence of at least 12 contiguous nucleotides belonging to SEQ ID NO: 4 or to the sequence complementary thereto, or a product of transcription of said fragment.

4. The fragment or transcription product as claimed in claim 3, wherein the fragment comprises a sequence of at least 15 contiguous nucleotides belonging to SEQ ID NO: 4 or to a sequence complementary thereto.

5. The fragment or transcription product as claimed in claim 3, wherein the fragment comprises a sequence of at least 18 contiguous nucleotides belonging to SEQ ID NO: 4 or to the sequence complementary thereto.

6. The fragment or transcription product as claimed in claim 3, wherein the fragment comprises a sequence of at least 20, 21, 22, 23, 24, 27, 30, 33, 36, 39, 42, 45, 48, 51 or 54 contiguous nucleotides belonging to SEQ ID NO: 4 or to the sequence complementary thereto.

7. A DNA nucleotide fragment, wherein the fragment comprises a nucleotide sequence which, over at least 12 contiguous nucleotides, exhibits at least 90% identity, preferably at least 95% identity, and advantageously at least 98% or 99% identity, with SEQ ID NO: 4 or with the sequence complementary to SEQ ID NO: 4, with the exclusion of the sequences TAGTCGAGACTCAACCATCGC (SEQ ID NO: 38) and CCCGCCCCGCTGATGAAAAG (SEQ ID NO: 31) and of the nucleotide sequences complementary to said sequences, or a product of transcription of said fragment.

8. A DNA nucleotide fragment or transcription product as claimed in claim 7, wherein the fragment comprises a nucleotide sequence which, over at least 15 contiguous nucleotides, exhibits at least 90% identity, preferably at least 95% identity, and advantageously at least 98% or 99% identity, with SEQ ID NO: 4 or with the sequence complementary to SEQ ID NO: 4, with the exclusion of the sequences TAGTCGAGACTCAACCATCGC (SEQ ID NO: 38) and CCCGCCCCGCTGATGAAAAG (SEQ ID NO: 31) and of the nucleotide sequences complementary to said sequences.

9. A DNA nucleotide fragment or transcription product as claimed in claim 7, wherein the fragment comprises a nucleotide sequence which, over at least 18 contiguous nucleotides, exhibits at least 90% identity, preferably at least 95% identity, and advantageously at least 98% or 99% identity, with SEQ ID NO: 4 or with the sequence complementary to SEQ ID NO: 4, with the exclusion of the sequences TAGTCGAGACTCAACCATCGC (SEQ ID NO: 38) and CCCGCCCCGCTGATGAAAAG (SEQ ID NO: 31) and of the nucleotide sequences complementary to said sequences.

10. A DNA nucleotide fragment or transcription product as claimed in claim 7, wherein the fragment comprises nucleotide sequence which, over at least 20, 21, 22, 23, 24, 27, 30, 33, 36, 39, 42, 45, 48, 51 or 54 contiguous nucleotides, exhibits at least 90% identity, preferably at least 95% identity, and advantageously at least 98% or 99% identity, with SEQ ID NO: 4 or with the sequence complementary to SEQ ID NO: 4, with the exclusion of the sequences TAGTCGAGACTCAACCATCGC (SEQ ID NO: 38) and CCCGCCCCGCTGATGAAAAG (SEQ ID NO: 31) and of the nucleotide sequences complementary to said sequences.

11. The fragment or transcription product as claimed in claim 3, wherein said contiguous nucleotides belong to the segment beginning at nucleotide 2 and ending at nucleotide 286 of SEQ ID NO: 4, or a fragment complementary to said fragment.

12. The fragment or transcription product as claimed in claim 3, wherein said contiguous nucleotides belong to the segment beginning at nucleotide 4 and ending at nucleotide 144 of SEQ ID NO: 4, or a fragment complementary to said fragment.

13. The fragment or transcription product as claimed in claim 3, characterized in that wherein said contiguous nucleotides belong to the segment beginning at nucleotide 180 and ending at nucleotide 1004 of SEQ ID NO: 4, or a fragment complementary to said fragment.

14. The fragment or transcription product as claimed in claim 3, wherein said contiguous nucleotides belong to the segment beginning at nucleotide 614 and ending at nucleotide 820 of SEQ ID NO: 4, or a fragment complementary to said fragment.

15. The fragment or transcription product as claimed in claim 3, wherein said contiguous nucleotides belong to the segment beginning at nucleotide 1228 and ending at nucleotide 1314 of SEQ ID NO: 4, or a fragment complementary to said fragment.

16. The fragment or transcription product as claimed in claim 3, wherein said contiguous nucleotides belong to the segment beginning at nucleotide 1283 and ending at nucleotide 1197 of the sequence complementary to SEQ ID NO: 4, or a fragment complementary to said fragment.

17. The fragment or transcription product as claimed in claim 3, wherein said contiguous nucleotides belong to the segment beginning at nucleotide 1264 and ending at nucleotide 1067 of the sequence complementary to SEQ ID NO: 4, or a fragment complementary to said fragment.

18. The fragment or transcription product as claimed in claim 3, wherein said contiguous nucleotides belong to the segment beginning at nucleotide 1209 and ending at nucleotide 1099 of the sequence complementary to SEQ ID NO: 4, or a fragment complementary to said fragment.

19. The fragment or transcription product as claimed in claim 3, wherein said contiguous nucleotides belong to the segment beginning at nucleotide 819 and ending at nucleotide 736 of the sequence complementary to SEQ ID NO: 4, or a fragment complementary to said fragment.

20. The fragment or transcription product as claimed in claim 3, wherein said contiguous nucleotides belong to the segment beginning at nucleotide 800 and ending at nucleotide 6 of the sequence complementary to SEQ ID NO: 4, or a fragment complementary to said fragment.

21. The fragment or transcription product as claimed in claim 3, wherein said contiguous nucleotides belong to the segment beginning at nucleotide 784 and ending at nucleotide 629 of the sequence complementary to SEQ ID NO: 4, or a fragment complementary to said fragment.

22. The fragment or transcription product as claimed in claim 3, wherein said contiguous nucleotides belong to the segment beginning at nucleotide 610 and ending at nucleotide 410 of the sequence complementary to SEQ ID NO: 4, or a fragment complementary to said fragment.

23. The fragment or transcription product as claimed in claim 3, wherein said contiguous nucleotides belong to the segment beginning at nucleotide 391 and ending at nucleotide 221 of the sequence complementary to SEQ ID NO: 4, or a fragment complementary to said fragment.

24. The fragment or transcription product as claimed in claim 3, wherein it comprises or consists of any one of the sequences SEQ ID NOS: 5 to 17 or any one of the sequences complementary to sequences SEQ ID NOS: 5 to 17.

25. A product of transcription of a sequence as defined in claim 1.

26. A DNA molecule, comprising a sequence as defined in claim 1.

27. An RNA molecule, comprising a product of transcription of a DNA molecule as defined in claim 26.

28. A polypeptide whose polypeptide sequence is encoded by a fragment as defined in claim 7.

29. The polypeptide as claimed in claim 28, whose polypeptide sequence comprises any one of the sequences SEQ ID NOS: 18 to 30 or of a polypeptide sequence equivalent to any one of the sequences SEQ ID NOS: 18 to 30, in which (i) the amino acids alanine, proline and glycine are equivalents, (ii) the amino acids aspartic acid and glutamic acid are equivalents, (iii) the amino acids histidine, lysine and arginine are equivalents, (iv) the amino acids asparagine, glutamine, serine and threonine are equivalents, (v) the amino acids phenylalanine, tyrosine and tryptophan are equivalents, and (vi) the amino acids isoleucine, leucine, valine and methionine are equivalents.

30. The polypeptide fragment as claimed in claim 28, comprising a peptide sequence of at least 4, 5, 6, 7, 8, 9, 10, 11, 12, 13, 14, 15, 16, 17 or amino acids belonging to any one of the sequences SEQ ID NOS: 18 to 30 or to a sequence equivalent to any one of the sequences SEQ ID NOS: 18 to 30, in which (i) the amino acids alanine, proline and glycine are equivalents, (ii) the amino acids aspartic acid and glutamic acid are equivalents, (iii) the amino acids histidine, lysine and arginine are equivalents, (iv) the amino acids asparagine, glutamine, serine and threonine are equivalents, (v) the amino acids phenylalanine, tyrosine and tryptophan are equivalents, and (vi) the amino acids isoleucine, leucine, valine and methionine are equivalents.

31. An epitope, comprising a peptide sequence of at least 6, 8, 9, 10, 12, 15 or 18 amino acids, and at most of 10, 12, 15 or 18 amino acids, in particular in that its sequence consists of a peptide sequence of 6 to 10 amino acids, of 6 to 12 amino acids, of 6 to 15 amino acids, of 6 to 18 amino acids, of 8 to 10 amino acids, of 8 to 12 amino acids, of 8 to 15 amino acids, of 8 to 18 amino acids and of 15 to 18 amino acids, of any one of the sequences represented in SEQ ID NOS: 18 to 30 or of a polypeptide sequence functionally equivalent to said sequences SEQ ID NOS: 18 to 30.

32. An expression cassette that is functional in a cell derived from a prokaryotic or eukaryotic organism, allowing the expression of a nucleic acid sequence as claimed in claim 1, placed under the control of the elements required for its expression.

33. A vector comprising an expression cassette as claimed in claim 32.

34. A cell derived from a eukaryotic or prokaryotic organism comprising an expression cassette as claimed in claim 32 or an expression vector comprising said expression cassette.

35. The cell as claimed in claim 34, characterized in that it is derived from a eukaryotic organism, in particular cells originating from animals such as mammals, reptiles or insects, preferably cells chosen from COS, CHO, Vero, BHK, PK 15 and RK 13 cells; human osteosarcoma cell lines, HeLa human cell lines and human hepatoma cell lines; insect cell lines.

36. The cell as claimed in claim 34, characterized in that it is derived from a lower eukaryotic organism, in particular derived from yeast such as Saccharomyces, Schizosaccharomyces, Kluveromyces, Hanseluna, Yarowia, Schwaniomyces, Zygosaccharomyces and Pichia, and preferably chosen from Saccharomyces cerevisiae, Saccharomyces carlsbergensis, Schizosaccharomyces pombe, Kluveromyces lactis and Pichia pastoris cells.

37. The cell as claimed in claim 34, characterized in that it is derived from a prokaryotic organism, preferably E. coli.

38. A polypeptide that can be produced by an expression cassette as claimed in claim 32.

39. A method for preparing a polypeptide whose peptide sequence is encoded by a nucleotide sequence as claimed in claim 1, according to which a host cell is cultured in an appropriate culture medium, and said polypeptide produced is purified, to a required degree of purity, wherein said host cell is derived from a prokaryotic or eukaroytic organism and comprises an expression cassette that allows the expression of said nucleotide sequence, placed under the control of the elements required for its expression.

40. An immunogenic polypeptide, comprising a polypeptide as defined in claim 28.

41. A monoclonal or polyclonal antibody that can be obtained by immunization of a mammalian animal with an immunogenic polypeptide as defined in claim 40.

42. A diagnostic composition, comprising a polypeptide as defined in claim 28.

43. A diagnostic composition, comprising a monoclonal antibody or a polyclonal antibody as defined in claim 41.

44. A method for detecting antibodies directed against the HXHV virus, according to which a biological sample from a patient suspected of being infected with HXHV virus is brought into contact with a diagnostic composition as defined in claim 42, under predetermined conditions which allow the formation of antibody/antigen complexes, and the formation of said complexes is detected.

45. A method for detecting a polypeptide as defined in claim 28, in a biological sample from a patient suspected of being infected with the HXHV virus, according to which the biological sample is brought into contact with a diagnostic composition comprising a monoclonal or polyclonal antibody that can be obtained by immunization of a mammalian animal with an immunogenic polypeptide comprising or consisting of a polypeptide whose peptide sequence is encoded by SEQ ID NO: 4 or the sequence complementary to SEQ ID NO: 4, under predetermined conditions which allow the formation of antibody/antigen complexes, and the formation of said complexes is detected.

46. An immunogenic or vaccine composition, comprising a polypeptide as defined in claim 28, combined with an appropriate vehicle and/or adjuvant and/or diluent and/or with a pharmaceutically acceptable excipient.

47. A probe of at least 12 nucleotides, wherein the probe is capable of hybridizing to a nucleic acid sequence as defined in claim 2, the hybridization being carried out under given stringency conditions.

48. A primer of at least 12 nucleotides, wherein the primer is capable of hybridizing to a nucleic acid sequence as defined in claim 2, the hybridization being carried out under given stringency conditions.

49. The primer as claimed in claim 48, wherein the primer is chosen from the primers SEQ ID NOS: 32 to 37.

50. A pair of primers as claimed in claim 48, chosen from one of the following pairs: SEQ ID NO: 31/SEQ ID NO: 32, SEQ ID NO: 31/SEQ ID NO: 33, SEQ ID NO: 34/SEQ ID NO: 35, and SEQ ID NO: 36/SEQ ID NO: 37.

51. An anti-nucleic acid antibody, wherein the anti-nucleic acid antibody is capable of binding to a nucleic acid sequence as defined in claim 2.

52. (canceled)

53. A method for detecting a viral DNA or RNA, in a biological sample from a patient suspected of being infected with the HXHV virus, according to which said sample is, if necessary, treated so as to extract the DNA or the RNA therefrom, said DNA or RNA is brought into contact with at least one probe as defined in claim 47, under given stringency conditions, and the presence of viral DNA or RNA in the sample is detected by demonstrating hybridization of said viral DNA or RNA with said at least one probe.

54. A method for detecting viral DNA and/or RNA of the HXHV virus, according to which a biological sample such as serum, plasma or blood is taken from a patient, said sample is, if necessary, treated so as to extract the DNA and/or the RNA therefrom, said sample is brought into contact with at least one anti-nucleic acid antibody as defined in claim 51, said antibody being optionally labeled with any appropriate label, and the formation of a nucleic acid/antibody complex is demonstrated.

55. A vaccine composition comprising a DNA sequence encoding at least one polypeptide as defined in claim 28, said DNA being mixed with a pharmaceutically acceptable vehicle and/or diluent and/or excipient.

56. A vector comprising at least one gene of therapeutic or vaccine interest, said gene encoding in particular at least one polypeptide as defined in claim 28.

57. A therapeutic or vaccine composition, comprising a vector as defined in claim 56 and in that said gene of interest is placed under the control of elements that ensure its expression in vivo.

58. A genetically modified cell, in particular chosen from eukaryotic cells, such as COS, CHO, Vero, BHK, PK 15 and RK 13 cells; human osteosarcoma cell lines, HeLa human cell lines and human hepatoma cell lines, insect cell lines; cells of lower eukaryotes, such as yeast cells, in particular cells derived from Saccharomyces, Schizosaccharomyces, Kluveromyces, Hanseluna, Yarowia, Schwaniomyces, Zygosaccharomyces and Pichia, and preferably chosen from Saccharomyces cerevisiae, Saccharomyces carlsbergensis, Schizosaccharomyces pombe, Kluveromyces lactis and Pichia pastoris cells; prokaryotic cells, such as those derived from E. coli; said cells being transformed with at least one nucleic acid sequence as claimed in claim 1.

59. A pharmaceutical or vaccine composition, comprising a cell as claimed in claim 58.

60. A polypeptide whose peptide sequence is encoded by a sequence as defined in claim 1.

61. A polypeptide whose peptide sequence is encoded by a fragment as defined in claim 3.

62. A method for detecting a viral DNA or RNA, in a biological sample from a patient suspected of being infected with the HXHV virus, according to which said sample is, if necessary, treated so as to extract the DNA or RNA therefrom, said DNA or RNA is brought into contact with at least one primer according to claim 48, under given stringency conditions, and the presence of viral DNA or RNA is the sample is detected by amplifying said DNA or RNA using said at least one primer.
